(19) United States
(12) Patent Application Publication
(10) Pub. No.: US 2025/0339201 A1
(43) Pub. Date: Nov. 6, 2025

(54) Radio-Frequency Ablation and Direct Current Electroporation Catheters

(71) Applicant: St. Jude Medical, Cardiology Division, Inc., St. Paul, MN (US)

(72) Inventors: Derek C. Sutermeister, Ham Lake, MN (US); Troy T. Tegg, Elk River, MN (US); Salo Arias, Brooklyn Park, MN (US)

(73) Assignee: St. Jude Medical, Cardiology Division, Inc., St. Paul, MN (US)

(21) Appl. No.: 19/213,602

(22) Filed: May 20, 2025

Related U.S. Application Data
(63) Continuation of application No. 17/502,902, filed on Oct. 15, 2021, now Pat. No. 12,376,901.
Application No. 17/502,902 is a continuation of application No. 16/418,296, filed on May 21, 2019, now abandoned.
(60) Provisional application No. 62/674,314, filed on May 21, 2018.

(51) Int. Cl. A61B 18/14; A61B 5/00; A61B 5/287; A61B 18/00

(52) U.S. Cl. CPC A61B 18/1492; A61B 5/287; A61B 5/6858; A61B 2018/00267; A61B 2018/00351; A61B 2018/00577; A61B 2018/00613; A61B 2018/00839; A61B 2018/1405; A61B 2018/1467; A61B 2562/0209

(57) ABSTRACT

Aspects of the present disclosure are directed to flexible catheters for both electrophysiology mapping and ablation using a high-density array of electrodes. These catheters may be used to detect electrophysiological characteristics of tissue in contact with the electrodes, and conduct monopolar and bipolar ablations of the tissue.

### CROSS-REFERENCES TO RELATED APPLICATIONS

The present application is a Continuation of U.S. patent application Ser. No. 17/502,902 filed Oct. 15, 2021 (Allowed); which is a Continuation of U.S. patent application Ser. No. 16/418,296 filed May 21, 2019; which claims the benefit of U.S. Provisional Appln. No. 62/674,314 filed May 21, 2018; the full disclosures which are incorporated herein by reference in their entirety for all purposes.

### BACKGROUND

Field. The instant disclosure relates to radio-frequency ablation catheters for treating myocardial tissue within a cardiac muscle, for example. In particular, the instant disclosure relates to basket and planar array catheters including a plurality of electrodes positioned in a high-density array.

Background Art. Catheters have been used for cardiac medical procedures for many years. Catheters can be used, for example, to diagnose and treat cardiac arrhythmias, while positioned at a specific location within a body that is otherwise inaccessible without a more invasive procedure.

Conventional ablation catheters may include, for example, a plurality of adjacent ring electrodes encircling the longitudinal axis of a basket catheter, for example. The ring electrodes may be constructed from platinum or some other metal. These ring electrodes are relatively rigid, and may deliver an ablation therapy (e.g., RF ablation energy) to treat symptoms related to, for example, a cardiac arrhythmia.

When conducting an ablation therapy on myocardial tissue, the beating of the heart, especially if erratic or irregular, makes it difficult to keep adequate contact between electrodes and tissue for a sufficient length of time. These problems are exacerbated on contoured, irregular, or trabeculated surfaces. If the contact between the electrodes and the tissue cannot be sufficiently maintained, quality lesions are unlikely to result.

Typically, cardiac ablation therapies are conducted using a focal point ablation catheter. Focal point ablation catheters deliver energy between a single electrode and a ground pad. As electrophysiology mapping becomes more precise, ablation therapies may likewise be more targeted. More targeted ablation therapies will limit unnecessary tissue damage.

Ablation therapies, such as for atrial fibrillation, have extended durations as the clinician must introduce an electrophysiology mapping catheter into the patient's left atrium, confirm the diagnosis, and determine an ablation therapy strategy before removing the electrophysiology mapping catheter. An ablation catheter is then introduced to complete the ablation therapy, followed by reintroduction of the electrophysiology mapping catheter to confirm the efficacy of the therapy. In view of the foregoing, a catheter capable of both electrophysiology mapping and ablation therapy would be desirable to limit the duration of the operation.

The foregoing discussion is intended only to illustrate the present field and should not be taken as a disavowal of claim scope.

### BRIEF SUMMARY

Aspects of the present disclosure are directed to flexible catheters for both electrophysiology mapping and ablation using a high-density array of electrodes. These catheters may be used to detect electrophysiological characteristics of tissue in contact with the electrodes, and conduct monopolar and/or bipolar ablations of the tissue. In particular, the instant disclosure relates to both planar and basket-type end effectors coupled to a distal end of a catheter shaft.

Several embodiments of the present disclosure are directed to a planar array catheter including an elongated catheter shaft and a flexible, planar array coupled to a distal end of the catheter shaft. The elongated catheter shaft defines a longitudinal axis. The flexible, planar array conforms to tissue, and includes two or more struts extending substantially parallel with the longitudinal axis. Each of the struts lay in a common plane and have a plurality of electrodes coupled thereto. The plurality of electrodes detect electrophysiological characteristics of tissue in contact with the planar array and selectively ablate the tissue. In more specific embodiments, the plurality of electrodes in the planar array may operate in both monopolar and bipolar configurations for tissue ablation.

Various embodiments of the present disclosure are directed to basket catheters including an elongated catheter shaft with proximal and distal ends, a flexible basket with a plurality of splines, and a plurality of electrodes mounted to the spline. The flexible basket coupled to the distal end of the catheter shaft and conforming to tissue. The plurality of electrodes detect electrophysiological characteristics of tissue in contact with the basket and selectively ablate the tissue. In some specific embodiments, the basket catheter further includes a plurality of temperature sensors, and ablation controller circuitry. Each of the temperature sensors are mechanically coupled to the splines and placed in thermal communication with at least one of the electrodes. The ablation controller circuitry is communicatively coupled to the plurality of temperature sensors and the plurality of electrodes. The ablation controller circuitry controls the power delivery to each electrode based at least in part upon the temperature measured in proximity to each electrode by the temperature sensors.

The foregoing and other aspects, features, details, utilities, and advantages of the present disclosure will be apparent from reading the following description and claims, and from reviewing the accompanying drawings.

### BRIEF DESCRIPTION OF THE DRAWINGS

Various example embodiments may be more completely understood in consideration of the following detailed description in connection with the accompanying drawings, in which:

FIG. 1 is a diagrammatic overview of an electrophysiology catheter system, consistent with various embodiments of the present disclosure;

FIG. 2A is an isometric side view of a basket end effector of an electrophysiology catheter, consistent with various embodiments of the present disclosure;

FIG. 2B is a close-up view of a portion of four adjacent splines of the basket end effector of FIG. 2A, consistent with various embodiments of the present disclosure;

FIG. 2C is a close-up view of a portion of two adjacent splines of the basket end effector of FIG. 2A and a ground pad which together form a radio-frequency ablation system, consistent with various embodiments of the present disclosure;

FIG. 3A is a top view of a planar end effector of an electrophysiology mapping catheter, consistent with various embodiments of the present disclosure;

FIG. 3B depicts the planar array catheter of FIG. 3A with an array of electrodes contacting tissue, consistent with various embodiments of the present disclosure; and

FIG. 3C depicts the planar array catheter of FIG. 3A overlaying vasculature, consistent with various embodiments of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are directed to flexible catheters for both electrophysiology mapping and ablation using a high-density array of electrodes. These catheters may be used to detect electrophysiological characteristics of tissue in contact with the electrodes, and conduct monopolar and/or bipolar ablations of the tissue. In particular, the instant disclosure relates to both planar and basket-type end effectors coupled to a distal end of a catheter shaft.

To conduct an electrophysiology mapping of a cardiac muscle, pacing is conducted. During the pacing procedure, adjacent electrodes are assigned to bipole pairings, and each bipole pair samples the electrical characteristics of the tissue between the pair. The resulting electrical signals are received and processed by controller circuitry. The controller circuitry develops an electrophysiology mapping by associating the signal samples from each bipole pair with a location of the tissue sampled by the bipole pair. The electrogram from each bipole pair may be analyzed and various electrical characteristics may be visually indicated on an electrophysiology map by color-coding (or other visual indication scheme, e.g., shading, patterning, etc.). In some embodiments, the color-coding may be based on the electrogram voltage at each location (e.g., mean, average, max, etc.). In other embodiments, the number of times the electrical signal exceeds a threshold voltage (or a voltage slope changes signs) during a sampling window may be visually displayed on the map. In yet other embodiments, total energy sampled during a time window may be displayed. Various other methods of fractionation accounting are known, and may be used as one or more factors of the resulting color-code displayed on the electrophysiology map. These electrophysiology maps may be used by a clinician to verify a diagnosis, provide insight into a desired ablation therapy strategy, and to verify the efficacy of the therapy.

Aspects of the present disclosure are directed to intravascular catheters with end effectors capable of electrophysiology mapping and mono/bipolar radio-frequency ablation treatment. Historically, cardiac ablation therapy has been conducted using point-by-point ablation techniques, delivering energy between a single electrode positioned on the distal tip of the catheter and a ground pad electrically coupled to the patient's chest. However, high-density electrophysiology mapping catheters have facilitated improved diagnostic specificity, and thereby a clinician may use the electrophysiology maps to more precisely target an ablation therapy to problematic tissue (e.g., such as tissue containing arrhythmic foci). This is particularly desirable as a clinician wishes to minimize the ablation of healthy myocardial tissue as much as possible, to maintain healthy functionality of the left atrium. To further improve ablation therapy workflow, aspects of the present disclosure are directed to using a single catheter to conduct both the electrophysiology mapping of the left atrium, as well as the ablation therapy. By combining such functionality into a single catheter, length of an ablation therapy (and operating room time) may be reduced. More specific embodiments of the present disclosure are directed to controlling ablation depth of an ablation catheter. Such embodiments are facilitated by improved three-dimensional electrophysiology mapping, which indicate the electrophysiology characteristics of the contacted myocardial tissue sub-surface. The ablation therapy may then be customized to provide depth-varying tissue ablation therapy throughout the left atrium using a combination of monopolar and bipolar type radio-frequency tissue ablation.

In many adults, myocardial tissue depth is typically less than 3 millimeters, and often less than 2 millimeters. Aspects of the present disclosure are directed to customizing a patient's tissue ablation therapy, to alleviate symptoms related to a cardiac arrhythmia for example, by varying ablation therapy treatment depths and to only treat compromised tissue. For example, an ablation therapy treatment plan may use a combination of monopolar RF (ablating between a single electrode and a ground pad) and bipolar RF modes (ablating between electrodes on the catheter) to vary an ablation treatment depth. Such variable-depth ablation therapy treatment mitigates risk to susceptible tissue such as the phrenic nerve. In more specific embodiments, multiplexing or selected sequential energy delivery for lesion formation may be utilized to further customize the ablation therapy.

In some specific aspects of the present disclosure, a basket catheter including 8 splines is disclosed. Each of the splines is comprised of a shape memory material which returns to a semi-circular shape upon exiting an introducer. Each of the splines is equally distributed circumferentially about the basket relative to the other splines. When expanded, the 8 splines form a substantially circular-shaped basket. Each of the splines includes a row of electrodes extending along a length of the splines. The electrodes may be evenly distributed along the length of the splines, or unevenly distributed along the length of the splines for specialized applications. For example, the distribution of the electrodes may be weighted toward a distal end of the basket where the basket catheter is intended, for example, to diagnose cardiac arrhythmias. Many cardiac arrhythmias are triggered by stray electrical signals emanating from one or more of the pulmonary veins. Assuming a transseptal approach to the left atrium, the distal end of the basket, including its high-density array of electrodes, would be orientated by a clinician with the pulmonary veins. Once in place within the left atrium, the basket catheter is capable of conducting an electrophysiology mapping of the left atrium, ablating myocardial tissue in proximity to the pulmonary veins to alleviate symptoms related to atrial fibrillation, and re-mapping the left atrium to verify the efficacy of the therapy.

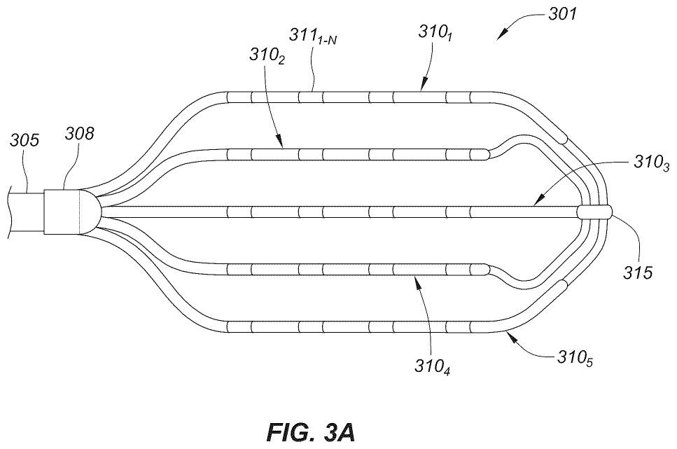

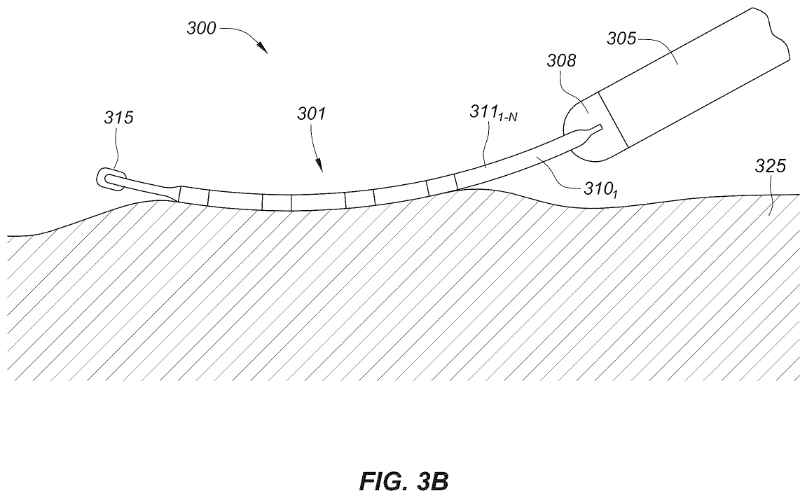

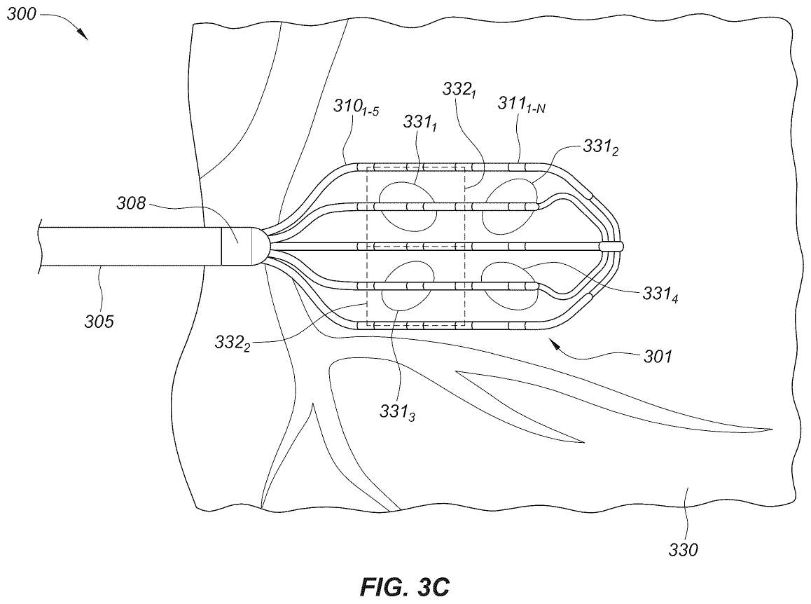

In some specific aspects of the present disclosure, a planar array catheter including five struts is disclosed. Each of the struts may be aligned with, and extend parallel to, a longitudinal axis of the catheter shaft. Each strut is coupled to the other struts of the planar array at proximal and distal ends. The struts each include a row of electrodes extending along a length of the struts. In some specific embodiments, the electrodes are evenly distributed along the length of the struts and between adjacent struts of the planar array. According to various embodiments, the planar catheter array of the present disclosure may include at least four struts, five struts, six struts, seven struts, or perhaps even eight struts. In the embodiment illustrated in FIG. 3A, the array contains five struts.

The electrodes disclosed herein may be ring electrodes, and/or printed (spot) electrodes on substrates (e.g., flexible circuit boards). Advantageously, printed electrodes may be spaced more closely than ring electrodes. In some embodiments, for example, printed electrodes spaced 0.1 mm apart have been successfully deployed in a planar array catheter. More typically, ring electrodes and printed electrodes have been advantageously spaced 0.5 mm to 4 mm apart. It has been found that such electrode spacing facilitates desirable electrophysiology mapping granularity in a number of cardiovascular applications, for example. Moreover, high-density positioning of electrodes about a planar array or basket catheter may facilitate customizable ablation therapies which minimize the amount of lesioned tissue necessary to alleviate the effects of cardiac arrhythmias, such as atrial fibrillation, on a patient.

Conventional mapping catheter designs employ bipole electrode configurations to detect, measure, and display electrical signals from the heart, and point-by-point ablation catheters with monopole electrode configurations to facilitate tissue ablation. However, various aspects of the present disclosure are directed to using a combination of monopolar and bipolar configurations on the catheter to facilitate treatment of, for example, atrial fibrillation. The relative selection of monopole or bipole ablation treatment at a given tissue location may be based, for example, on the desired ablation depth or width. In some specific embodiments, ablation controller circuitry may receive an electrophysiology map of a target tissue area and determine the type of ablation therapy each tissue region within a target tissue area will receive. Alternatively, a clinician may manually design the ablation therapy based on an electrophysiology map provided, or otherwise approve/modify the treatment strategy designed by the ablation controller circuitry.

A basket catheter for ablation therapy, consistent with the present disclosure, may include a plurality of electrodes distributed about one or more of the splines which form the basket. Each of the electrodes may operate in a monopole or bipole configuration, or in both configurations simultaneously. That is, a single electrode may transmit radio-frequency energy to an adjacent electrode on the basket catheter and a patch electrode on a patient's chest simultaneously. In some more specific embodiments, a thermocouple may be placed beneath (or otherwise in close proximity to) one or more of the electrodes to enable temperature controlled radio-frequency tissue ablation.

Details of the various embodiments of the present disclosure are described below with specific reference to the figures.

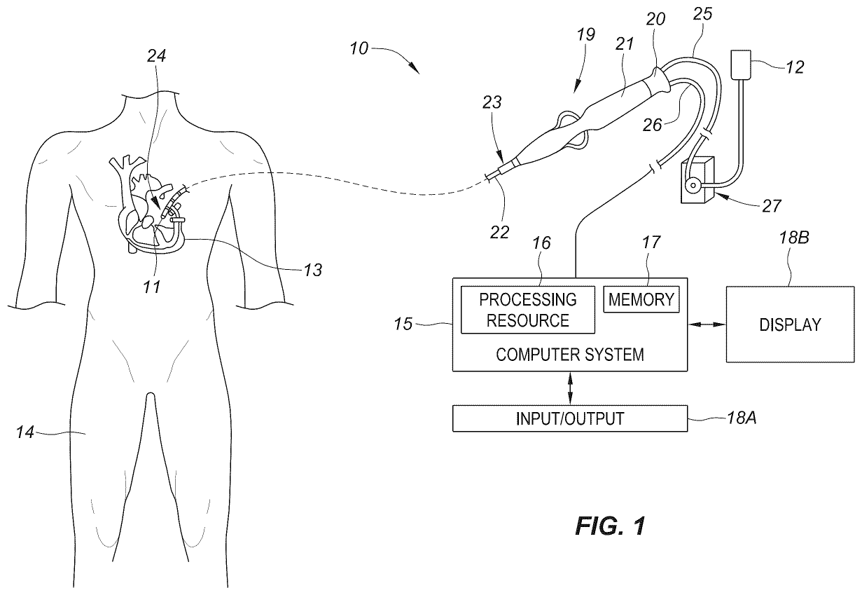

FIG. 1 is a diagrammatic overview of an electrophysiology catheter system, consistent with various embodiments of the present disclosure.

Referring now to the drawings wherein like reference numerals are used to identify identical components in the various views, FIG. 1 generally illustrates an electrophysiology catheter system 10 for force detecting having an elongated medical device 19 that includes a sensor assembly 11 (e.g., a plurality of electrodes for electrophysiology mapping and ablation) configured to be used in the body for medical procedures. The elongated medical device 19 may be used for diagnosis, visualization, and/or treatment of tissue 13 (such as cardiac or other tissue) in the body. For example, the medical device 19 may be used for ablation therapy of tissue 13 or mapping purposes in a patient's body 14. FIG. 1 further shows various sub-systems included in the overall system 10. The system 10 may include a main computer system 15 (including an electronic control unit 16 and data storage 17, e.g., memory). The computer system 15 may further include conventional interface components, such as various user input/output mechanisms 18A and a display 18B, among other components. Information provided by the sensor assembly 11 may be processed by the computer system 15 and may provide data to the clinician via the input/output mechanisms 18A and/or the display 18B, or in other ways as described herein.

In the illustrative embodiment of FIG. 1, the elongated medical device 19 may include a cable connector or interface 20, a handle 21, a tubular body or shaft 22 having a proximal end 23 and a distal end 24. The elongated medical device 19 may also include other conventional components not illustrated herein, such as a temperature sensor, additional electrodes, and corresponding conductors or leads. The connector 20 may provide mechanical, fluid and/or electrical connections for cables 25, 26 extending from a fluid reservoir 12 and a pump 27 and the computer system 15, respectively. The connector 20 may comprise conventional components known in the art and, as shown, may be disposed at the proximal end of the elongated medical device 19.

The handle 21 provides a portion for a user to grasp or hold the elongated medical device 19 and may further provide a mechanism for steering or guiding the shaft 22 within the patient's body 14. For example, the handle 21 may include a mechanism configured to change the tension on a pull-wire extending through the elongated medical device 19 to the distal end 24 of the shaft 22 or some other mechanism to steer the shaft 22. The handle 21 may be conventional in the art, and it will be understood that the configuration of the handle 21 may vary.

The computer system 15 may utilize software, hardware, firmware, and/or logic to perform a number of functions described herein. The computer system 15 may be a combination of hardware and instructions to share information. The hardware, for example may include processing resource 16 and/or a memory 17 (e.g., non-transitory computer-readable medium (CRM) database, etc.). A processing resource 16, as used herein, may include a number of processors capable of executing instructions stored by the memory resource 17. Processing resource 16 may be integrated in a single device or distributed across multiple devices. The instructions (e.g., computer-readable instructions (CRI)) may include instructions stored on the memory 17 and executable by the processing resource 16 for force detection.

The memory resource 17 is communicatively coupled with the processing resource 16. A memory 17, as used herein, may include a number of memory components capable of storing instructions that are executed by processing resource 16. Such a memory 17 may be a non-transitory computer readable storage medium, for example. The memory 17 may be integrated in a single device or distributed across multiple devices. Further, the memory 17 may be fully or partially integrated in the same device as the processing resource 16 or it may be separate but accessible to that device and the processing resource 16. Thus, it is noted that the computer system 15 may be implemented on a user device and/or a collection of user devices, on a mobile device and/or a collection of mobile devices, and/or on a combination of the user devices and the mobile devices.

The memory 17 may be communicatively coupled with the processing resource 16 via a communication link (e.g., path). The communication link may be local or remote to a computing device associated with the processing resource 16. Examples of a local communication link may include an electronic bus internal to a computing device where the memory 17 is one of a volatile, non-volatile, fixed, and/or removable storage medium in communication with the processing resource 16 via the electronic bus.

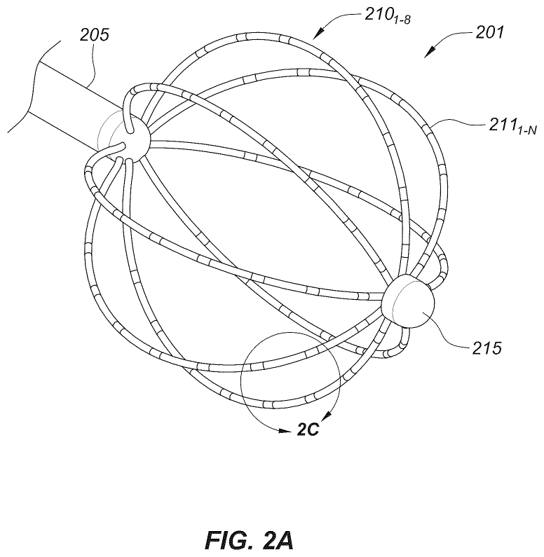

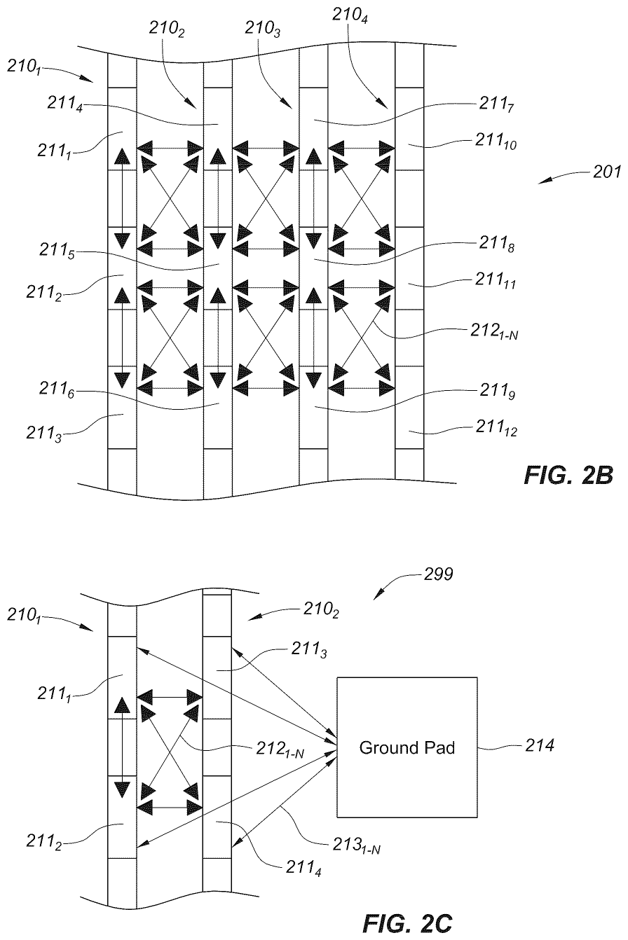

FIG. 2A is an isometric side view of a basket end effector (also referred to as a basket catheter) of an electrophysiology catheter, consistent with various embodiments of the present disclosure. The basket catheter 201 of FIG. 2A is shown in an expanded configuration. The basket 201 is comprised of a plurality of splines 210₁₋₈ which are coupled to a catheter shaft 205 at a proximal end and to a distal cap 215 (or one another) at a distal end. While the present embodiment presents a basket comprised of eight splines 210₁₋₈, basket catheters with three or more splines are readily envisioned, with the design depending on an intended clinical application and desired electrophysiology mapping granularity. To facilitate expansion/contraction of the basket, the splines 210₁₋₈ may be comprised of a shape-memory alloy (e.g., nitinol) which returns to a semi-circular shape after exiting an introducer. In yet other embodiments, the basket catheter may utilize a deployment member to expand/contract the basket.

In the present embodiment, each of the splines 210₁₋₈ includes a plurality of electrodes 211_{1-N} distributed about a length of each spline. While the embodiment presented in FIGS. 2A-C depicts electrode 211_{1-N} regularly distributed along the length of each spline, other embodiments may include unevenly distributed electrodes along the splines. For example, in pulmonary vein electrophysiology mapping applications, only a distal portion of the basket may be in contact with tissue proximal the pulmonary veins. Accordingly, a distribution of electrodes 211_{1-N} may be weighted toward a distal end of the basket 201 to facilitate enhanced electrophysiology mapping granularity in proximity to the pulmonary veins.

The electrodes 211_{1-N} may be used in various bipole configurations to facilitate measurement of electrical characteristics of tissue in contact with the electrodes. A first bipole pair may include a pair of electrodes 211 along a length of a spline 210, facilitating the collection of tissue electrical characteristic data in an orientation substantially parallel with the catheter's longitudinal axis. A second, orthogonal bipole pair may extend laterally across adjacent splines 210, facilitating the collection of tissue electrical characteristic data in an orientation substantially transverse to the catheter's longitudinal axis. To facilitate collecting this electrical data, these bipole electrode pairs may be independently addressable by signal processing circuitry. The signal processing circuitry analyzes the received signals from the various bipole electrode pairs to assemble a electrophysiology map which visualizes the electrophysiology data sensed by the basket catheter of the tissue in contact with the electrodes.

In various embodiments consistent with the present disclosure, splines 210 may be formed from flexible electronic circuit boards with each of the electrodes 211 coupled thereto and communicatively coupled to signal processing circuitry via electrical traces that extend along interior or exterior layers of the flexible printed circuit board. In some specific embodiments, each of the splines 210 may consist of nitinol. In such embodiments, the flex circuit may be either bonded directly to the nitinol, or, alternatively, the flex circuit may be directly bonded to Pebax™ tubing which houses the nitinol spline internally.

In some embodiments, the electrodes 211 may be 0.8 millimeters in diameter with a total surface area of 0.5 mm². The electrodes 211 on the basket catheter 201 need not be uniform in size and shape. For example, embodiments consistent with the present disclosure may include electrodes capable of electrophysiology mapping, RF tissue ablation, and optionally facilitating localization in an impedance or hybrid-based catheter navigation system (e.g., MediGuide™ System, and/or EnSite™ NavX™ System, each from Abbott).

While it may be desirable in some embodiments to have equal spacing between all of the electrodes 211 both on a spline 210 and between splines, knowledge of the relative spacing between each of the electrodes which form bipole pairs is sufficient to accurately capture electrical characteristic data of tissue in contact with the electrodes. In some specific embodiments, an edge-to-edge spacing for one or more of the bipole pairs of electrodes may be between 2-2.5 millimeters. In yet other specific embodiments, center-to-center spacing of the electrodes in a bipole pair may be between 0.5-4 millimeters.

In some specific embodiments, some of electrodes 211 on basket 201 may be multi-purpose, while other electrodes are single-purpose. For example, some of the electrodes may function as both navigation, ablation, and electrophysiology mapping electrodes, others may function only as electrophysiology mapping electrodes, and yet other electrodes may function only as navigation electrodes.

As further shown in FIG. 2A, a distal cap 215 may serve several purposes including coupling distal ends of the splines 210₁₋₈ back to one another (near a longitudinal axis of the catheter), and providing a distal most surface of the catheter that prevents unintentional trauma to tissue contacted therewith.

In various embodiments consistent with the present disclosure, each spline of the basket catheter may be coupled to one or more steering wires which when actuated expand and/or contract the splines to form the desired shape.

While the present disclosure is directed toward a basket catheter 201 with eight electrodes 211 on each spline 210, various other implementations are readily envisioned. For example, the basket catheter may include more or less splines and/or more or less electrodes on each respective spline.

As discussed in more detail below, one particular advantage of a basket catheter capable of both electrophysiology mapping and ablation therapy is reduction in surgery time as the clinician need not swap out the electrophysiology mapping catheter with an ablation catheter after confirming a treatment strategy. Moreover, the need for magnetic and/or impedance-based localization of the ablation catheter within the patient's cardiac muscle may be reduced as the relative location of target tissue for ablation is already known by virtue of the electrophysiology mapping and the static position of the basket catheter within the patient's left atrium.

FIG. 2B is a close-up view of a portion of four adjacent splines 210₁₋₄ of the basket 201 of FIG. 2A, consistent with various embodiments of the present disclosure. Each of the splines 210 include a number of electrodes 211₁₋₁₂ which may be used to sense the electrophysiological characteristics of tissue (often operating in a bipolar configuration with another adjacent electrode), and/or ablate tissue in contact therewith. The electrodes may ablate tissue using a bipolar configuration, or a uni-polar configuration where one or more of the electrodes are paired with a ground pad which is coupled to a patient's chest, for example. As shown in FIG. 2B, a number of bipolar electrode pairings 212_{1-N} are shown. These pairings may extend along a longitudinal axis of a spline, transverse to the longitudinal axis of the spline, or the electrode pairings may extend diagonally between two adjacent splines. Such a system may conduct electrophysiology mapping using a bipolar configuration of electrodes across a surface of a basket catheter, and/or conduct precise tissue ablation therapies which limit the necrosis of healthy tissue. For example, based on a generated electrophysiology map of tissue in a patient's left atrium, a bipolar ablation therapy may be implemented that ablates only tissue that is susceptible to transmitting stray electrical signals and/or myocardial tissue containing arrhythmic foci (which may generate such electrical signals).

One particular benefit of bipolar ablation therapy is that the actual energy delivered to target tissue is known, due to the close proximity of the positive and negative electrodes. Moreover, bipolar ablation therapy also limits energy delivery to non-target tissue by virtue of the relative proximity of the electrodes.

While FIG. 2B depicts bipole pairs of electrodes which are immediately adjacent to one another, other bipole pair arrangements are readily envisioned. For example, pairs of electrodes that are not immediately adjacent. For example, tissue ablation may be achieved to tissue in proximity to electrodes 211₁ and 211₁₂, when the electrodes are operated in a bipolar arrangement. In some embodiments a first number of electrodes (e.g., electrodes 211₁₋₃) on a first spline 210₁ may be operated in a bipolar arrangement with a second number of electrodes (e.g., electrodes 211₄₋₆) on a second spline 210₂. In yet further embodiments, a first number of electrodes (e.g., electrodes 211₁₋₃) on a first spline 210₁ may be operated in a bipolar arrangement with a third number of electrodes (e.g., electrodes 211₇₋₉) on a third spline 210₃. Further, a first number of electrodes (e.g., electrodes 211₁₋₃) on a first spline 210₁ may be operated in a bipolar arrangement with a fourth number of electrodes (e.g., electrodes 211₁₀₋₁₂) on a fourth spline 210₄.

FIG. 2C is a close-up view of a portion of two adjacent splines 210₁₋₂ of the basket catheter 201 of FIG. 2A and a ground pad 214 which together form a radio-frequency ablation system 299. As discussed in FIG. 2B, each spline 210 includes a number of electrodes 211₁₋₄. Each electrode may be paired with another adjacent electrode to facilitate bipolar electrophysiology mapping and/or tissue ablation (e.g., bipolar electrode pairings 212_{1-N}). Alternatively, or simultaneously, the electrodes 211₁₋₄ may also be paired with a ground pad 214 to operate in a monopolar ablation therapy configuration (e.g., monopolar electrode pairings 213_{1-N}). During an ablation therapy, electrodes operating in a monopolar configuration will achieve greater lesion depth, and bipolar configuration electrodes will create more precisely located lesions.

FIG. 3A is a top view of a planar array 301 of an electrophysiology mapping catheter, consistent with various embodiments of the present disclosure. The planar array 301 of the electrophysiology mapping catheter includes a high-density array of electrodes 311_{1-N}. The planar array 301 forms a flexible array of the electrodes 311_{1-N}. This array of electrodes is coupled to a flexible framework of struts 310₁₋₅ which extend along a plane that is substantially parallel with a longitudinal axis of catheter shaft 305. Each of the struts is precisely, laterally separated from each other to facilitate exact spacing between electrodes 311_{1-N} on adjacent struts 310₁₋₅, and the struts are coupled to one another at distal and proximal ends (e.g., at a distal tip 315 and bushing 308).

As shown in FIG. 3A, each of the five struts 310₁₋₅ may carry a plurality of electrodes 311, with the spacing of the electrodes along a length of the strut being the same (or at least known). Similarly, the spacing between electrodes 311 across struts 310 of the array may also be equal (or at least known). The result is a plurality of electrode bipole pairs with known spacing. For example, in some embodiments the center-to-center electrode spacing of a bipole pair may be between 0.5-4 mm. In yet more specific embodiments, the center-to-center electrode spacing of a bipole pair may be less than 0.5 millimeters (e.g., 0.1 mm). While the present embodiment is directed to bipole pairs with equal center-to-center spacing, various other embodiments of an electrode array consistent with the present disclosure may include an electrode array with equal edge-to-edge spacing. For example, in some embodiments the edge-to-edge electrode spacing may be between 0.5-4 mm. In yet more specific embodiments, the edge-to-edge electrode spacing may be less than 0.5 millimeters (e.g., 0.1 mm). Consideration of edge-to-edge spacing may be desirable where the electrodes 311 of the array 301 have different relative sizes (or surface areas).

Although the planar array 301 in FIG. 3A depicts five struts 310₁₋₅, the catheter may comprise more or less struts, with spacing between each respective strut based on a desired electrode spacing for a given electrophysiology application. Additionally, while the planar array 301 depicted in FIG. 3A shows 20 electrodes 311, the planar array may include more or fewer than 20 electrodes, and each strut need not have the same number of electrodes as adjacent struts.

In some embodiments, electrodes 311_{1-N} may be used in diagnostic, therapeutic, and/or mapping procedures. For example and without limitation, the electrodes 311 may be used for electrophysiological studies, pacing, cardiac mapping, and ablation. In some embodiments, the electrodes 311 may perform unipolar and/or bipolar tissue ablation therapy. The ablation therapy may create specific lines or patterns of lesions. In some embodiments, the electrodes 311 may receive electrical signals from a pacing electrode, which can be used for electrophysiological studies/mapping. Importantly, as the electrode spacing between adjacent electrodes on a strut 310, and those on adjacent struts, are the same (or otherwise known), bipole pairs with varying relative orientations may be sampled to determine electrical characteristics of the tissue in contact with the bipole pairs. In some embodiments, the electrodes 311 may perform a location or position sensing function related to localization (e.g., determine location and/or orientation of the catheter 301).

The planar array 301 is coupled to a distal end of a catheter shaft 305 at a bushing 308 (also referred to as a connector). The catheter shaft 305 may also define a catheter shaft longitudinal axis. In the present embodiment, each of the struts 310₁₋₅ extend parallel to the longitudinal axis. The catheter shaft 305 may be made of a flexible material, such that it can be threaded through a tortuous vasculature of a patient. In some embodiments, the catheter shaft 305 can include one or more ring electrodes disposed along a length of the catheter shaft. The ring electrodes may be used for diagnostic, therapeutic, localization and/or mapping procedures, for example. In one embodiment, planar array 301 may include one or more magnetic field sensors configured for use with an electromagnetic localization system such as the MediGuide™ System sold by St. Jude Medical, Inc. of St. Paul, Minnesota.

The planar array 301 may be adapted to conform to tissue (e.g., cardiac tissue). For example, when the planar array contacts tissue, each strut 310₁₋₅ may independently deflect to conform to the tissue. The ability for the planar array to deflect in response to tissue may be particularly beneficial when the planar array comes into contact with contoured, irregular, or trabeculated tissue. In some embodiments, the struts 310 (or the understructure of the struts) may be constructed from a flexible or spring-like material such as nitinol and/or a flexible substrate. The construction of the planar array struts 310₁₋₅ (including, for example, the length and/or diameter of the struts, and material) may be tailored to achieve desired resiliency, flexibility, foldability, conformability, and stiffness characteristics. Moreover, in some embodiments it may be desirable to vary one or more characteristics from the proximal end of a strut to the distal end of the strut, or between or among the plurality of struts forming the planar array 301. The collapsibility of materials such as nitinol and/or a flexible substrate provides the added benefit of facilitating insertion of the planar array into a delivery sheath or introducer, whether during delivery of the catheter into the body or removal of the catheter from the body at the end of a procedure.

Planar array catheters, including the high-density electrode array positioned thereon, may be used for, for example: (1) defining regional propagation maps of particularly sized areas on the walls of the heart; (2) identifying complex fractionated atrial electrograms for ablation; (3) identifying localized, focal potentials between the electrodes for higher electrogram resolution; and/or (4) more precisely targeting areas for ablation. Additionally, the catheters described herein may find application in epicardial and/or endocardial use, and more specifically for treating symptoms associated with Brugada syndrome. For example, the planar array embodiments depicted herein may be used in epicardial procedures where the planar array of electrodes is positioned between the myocardial surface and the pericardium. Alternatively, the planar array may be used in an endocardial procedure to sweep and/or analyze the inner surfaces of the myocardium and create high-density maps of the heart tissue's electrical properties.

While various embodiments of the planar array 301 disclosed in the present disclosure are depicted with ring electrodes 311_{1-N} coupled to the struts 310₁₋₅, embodiments with spot-type electrodes coupled to the struts are readily envisioned. Moreover, in yet further embodiments, the struts of the planar array may comprise flexible thin films compatible with printed circuit manufacturing techniques and/or have such thin films coupled to structural elements of the strut (e.g., nitinol-based structural elements). In such embodiments, spot-type electrodes may be printed onto the struts themselves. In flexible printed circuit embodiments of the present disclosure, the printed electrodes may be electrically coupled to signal processing circuitry and/or driver circuitry via traces extending on or within the one or more thin film layers. As many electrophysiology mapping applications require high signal fidelity, it is desirable to limit the transmission length of the analog signal, shield the transmission line itself, and/or convert the analog signal to a digital signal close to the source of the analog signal. Accordingly, aspects of the present disclosure are directed to placing signal processing circuitry (e.g., analog-to-digital converters, signal conditioning such as noise filtration and bandpass filters), and/or driver circuitry on the struts 310₁₋₅ or in close proximity thereto.

In embodiments of the planar array 301 including ring electrodes 311_{1-N}, the ring electrodes of the high-density electrode array may include the same type of electrode or a variety of various electrode types. For example, electrodes with smaller surface area may be used exclusively for electrophysiology mapping, while larger surface area electrodes may be used for mapping, tissue ablation, and/or localization. In some specific embodiments, the electrode array may include one or more slightly enlarged ring electrodes. These slightly enlarged electrodes may be used, for example, for more precise localization of the flexible array in mapping and navigation systems. It may also be possible to drive ablation current between these enlarged electrodes, if desired, for bipolar ablation, or, alternatively to drive ablation current in unipolar mode between one or more of these enlarged ring electrodes and, for example, a ground or patch electrode located on a patient (e.g., on the patient's back). Similarly, the electrodes 311_{1-N} in some embodiments may all be capable of performing unipolar or bipolar ablation therapies. Alternatively or concurrently, current may travel between one or more of the enlarged electrodes and any one, or all, of the electrodes. This unipolar or bipolar ablation therapy techniques may be used to create specific lesion lines or lesion patterns. As also seen in FIG. 3A, there may be a distal tip 315 where one or more of the struts 310₁₋₅ come together. This distal tip 315 may be constructed from metal or some other radiopaque material to provide fluoroscopy visualization. The distal tip 315 may further facilitate (semi-) independent planar movement between the struts 310₁₋₅.

In some embodiments of the present disclosure, the mapping catheter 301 may include steering wires which extend a length of catheter shaft 305. Prior to reaching a bushing 308 that couples the catheter shaft 305 to struts 310₁₋₅ of planar array 301, the steering wires may be coupled to steering rings which receive a tension from a proximal end of the steering wires and facilitates steering the catheter shaft 305 and the planar array 301 through a patient's vasculature. As further shown in FIG. 3A, each of the struts 310₁₋₅ includes a plurality of electrodes 311_{1-N} distributed along a length of the struts. In the present embodiment, each of the electrodes are equally spaced from each of the adjacent electrodes. When controller circuitry samples electrical signals from bipole pairs of electrodes within the planar array 301, each of the bipole pairs will detect various electrical characteristics indicative of the tissue health in contact with the electrodes. The five struts 310₁₋₅ are designed to maintain the electrodes 311_{1-N} in a spaced relationship so that each bipole pair of electrodes captures electrophysiology data of tissue across a known distance.

While many embodiments of the present disclosure are directed to electrophysiology mapping, embodiments of the present disclosure may also be configured for pacing (as well). For example, one or more electrodes 311_{1-N} may send pacing signals to, for example, cardiac tissue.

Though not shown in FIGS. 3A, various embodiments of the planar array catheter 301 may include one or more irrigation ports. For example, proximal irrigant port(s) may be located on/at the distal end of proximal bushing 308, the proximal irrigant port(s) positioned to deliver irrigant to or near the point where the electrode carrying struts 310₁₋₅ exit from the distal end of the proximal bushing that is mounted on the distal end of the catheter shaft 305 in this embodiment. In some more specific embodiments, second, distal irrigation port(s) may be located near the distal intersection of the struts 310₁₋₅ and on or near distal tip 315. In yet further embodiments, if desired, multiple irrigation ports could be present at various positions along the struts 310. Where more than one irrigant port is positioned at proximal and/or distal ends of the planar array 301, more uniform irrigant distribution at or near the proximal/distal apex of the struts 310 may be facilitated.

FIG. 3B depicts the planar array catheter 300 of FIG. 3A with an array 301 of electrodes 311_{1-N} contacting tissue 325. The tissue 305 in the present embodiment is depicted as trabeculated, irregular, or contoured tissue. As shown in FIG. 3B, the flexible struts of the planar array, including flexible strut 310₁, conforms to the tissue 325, enabling a physician to place the planar array 301 (and its electrodes 311_{1-N}) into constant contact with the tissue 325. Each strut 310₁₋₅ may independently deflect to conform to the tissue. As a result, the electrical signals (indicative of the tissue's electrical activity) sampled by the planar array exhibit enhanced accuracy, and thereby have improved diagnostic value. Each of the flexible struts include a plurality of electrodes 311_{1-N}, and are coupled to the other adjacent struts of the planar array 301 at distal member 315 and bushing 308. The bushing 308 further couples the planar array 301 to shaft 305.

FIG. 3C depicts the planar array catheter 300 of FIG. 3A overlaying vasculature 330, consistent with various embodiments of the present disclosure. In some embodiments of the present disclosure, the catheter 300 may include steering wires which extend a length of catheter shaft 305. Prior to reaching a bushing 308 that couples the catheter shaft 305 to struts 310₁₋₅ of planar array 301, the steering wires may be coupled to a pull ring which receives a tension from a proximal end of the steering wires and facilitates steering the catheter shaft 305 and the planar array 301 through a patient's vasculature. As further shown in FIG. 3C, each of the struts 310₁₋₅ includes a plurality of electrodes 311_{1-N} distributed along a length of the struts. In the present embodiment, each of the electrodes are equally spaced. When controller circuitry samples electrical signals from bipole pairs of electrodes within the planar array 301, each of the bipole pairs will detect various electrical characteristics indicative of the tissue health in contact with the electrodes.

In FIG. 3C, vasculature 330 is a left atrium of a cardiac muscle, with the planar array 301 extending across four pulmonary veins 331₁₋₄. For discussion purposes, an electrophysiology mapping of the patient's left atrium has been completed and a clinician has confirmed the patient's diagnosis of atrial fibrillation. Based upon the electrophysiology mapping taken in proximity to the pulmonary veins 331, the clinician has determined that stray electrical signals are emanating from right superior pulmonary vein 331₁ and right inferior pulmonary vein 331₃. Accordingly, the clinician has determined that the right superior and inferior pulmonary veins must be isolated from the left atrium to alleviate the patient's atrial fibrillation symptoms. A plurality of electrodes circumferentially surrounding each of the target pulmonary veins may then be selected and used in either or both mono/bipolar configuration to conduct tissue ablation about the pulmonary veins. The resulting lesions 332₁₋₂ each surround a respective pulmonary vein and exhibit electrical characteristics which inhibit electrical signal distribution within the left atrium of stray electrical signals from arrhythmic foci within the pulmonary veins.

While aspects of the present disclosure have been presented as being readily applicable to radio-frequency ablation techniques, aspects of the present disclosure are also readily applied to irreversible electroporation (also referred to as direct current ablation). Moreover, while bipolar and monopolar RF techniques have been disclosed herein, variations on such techniques are also envisioned. For example, a bipolar ablation configuration may include alternating adjacent electrode polarities on the electrode array with the ground pad having a negative polarization. In one monopolar ablation configuration, the ground pad may have an alternating polarity over time, with adjacent electrodes carrying alternating polarities. Further, aspects of the present disclosure have been discussed including diagnosis and treatment of cardiac arrhythmias (e.g., atrial fibrillation); however, the present disclosure is readily applicable to the diagnosis and treatment of a number of different ailments, for example, Brugada syndrome.

Yet further embodiments consistent with the present disclosure may be directed to high-voltage direct current (“DC”) ablation (either bi-polar or mono-polar configuration). In such embodiments the high-voltage DC may include voltages between 400 and 4,000 Volts, and minimized current draw to target a voltage gradient rather than current delivery.

U.S. Provisional Appln. No. 62/414,634 filed Oct. 28, 2016, U.S. Provisional Appln. No. 62/572,186 filed Oct. 13, 2017, and U.S. Pat. No. 11,172,858, are all generally directed to flexible, high-density mapping catheters and are incorporated by reference as though fully set forth herein.

While various embodiments of high-density electrode catheters are disclosed herein, the teachings of the present disclosure may be readily applied to various other catheter embodiments as disclosed, for example, in the following patents and patent applications which are hereby incorporated by reference: U.S. Provisional Appln. No. 61/753,429 filed Jan. 16, 2013; U.S. Provisional Appln. No. 60/939,799 filed May 23, 2007; U.S. Appln. No. 11/853,759 filed Sep. 11, 2007 (now U.S. Pat. No. 8,187,267); U.S. Provisional Appln. No. 60/947,791 filed Jul. 3, 2007; U.S. application Ser. No. 12/167,736 filed Jul. 3, 2008 (now U.S. Pat. No. 8,206,404); U.S. application Ser. No. 12/667,338 filed Jan. 20, 2011 (now U.S. Pat. No. 8,827,910); U.S. application Ser. No. 12/651,074 filed Dec. 31, 2009 (now U.S. Pat. No. 8,979,837); U.S. application Ser. No. 12/436,977 filed May 7, 2009 (now U.S. Pat. No. 11,395,694); U.S. application Ser. No. 12/723,110 filed Mar. 12, 2010 (now U.S. Pat. No. 8,734,440); U.S. Provisional Appln. No. 61/355,242 filed Jun. 16, 2010; U.S. application Ser. No. 12/982,715 filed Dec. 30, 2010 (now U.S. Pat. No. 8,974,454); U.S. application Ser. No. 13/159,446 filed Jun. 14, 2011 (now U.S. Pat. No. 10,220,187); PCT Appln. No. PCT/US2011/040629 filed Jun. 16, 2011 (published as WO 2011/159861 A2); U.S. application Ser. No. 13/162,392 filed Jun. 16, 2011 (published as US 2012/0010490 A1); U.S. application Ser. No. 13/704,619 filed Dec. 16, 2012 (now U.S. Pat. No. 10,118,015) which is a U.S. National Phase 371 of PCT/US2011/040781 filed Jun. 16, 2011 (published as WO 2011/159955 A1).

Various aspects of the present disclosure may be implemented in conjunction with OIS/OT-like signal processing algorithms for electrophysiology mapping. OIS/OT and related algorithms are discussed in more detail in U.S. Provisional Appln. No. 61/944,426 filed Feb. 25, 2014, U.S. application Ser. No. 15/118,522 filed Aug. 12, 2016 (now U.S. Pat. No. 10,470,682), and PCT Appln. No. PCT/US2014/011940 filed Jan. 16, 2014, which are hereby incorporated by referenced as though fully disclosed herein. Yet other embodiments of the present disclosure may be implemented in conjunction with various other algorithm types for electrophysiology mapping. For example, embodiments consistent with the present disclosure may utilize the electrode signal post-processing techniques, and electrophysiology mapping algorithms disclosed in the following publications, which are hereby incorporated by reference: Magtibay et al. JAHA 2017 (J Am Heart Assoc. 2017;6:e006447. DOI: 10.1161/JAHA.117.006447) (see, e.g., pages 6 and 7, and section titled “Omnipoles Provide the Largest Possible Bipolar Voltages”); and Haldar et al. Circulation AE 2017 (Circ Arrhythm Electrophysiol. 2017;10:e005018. DOI: 10.1161/CIRCEP.117.005018) (see, e.g., page 6, section titled “Omnipolar Voltage Amplitude Correlates to Largest Measurable Bipolar Vpp,” and FIG. 4).

Various embodiments presented herein are amenable to the application of spot electrodes coupled to a flexible electronic circuit, where the flexible electronic circuit may also (partially) comprise the splines and struts of the planar and basket catheters, respectively. Yet other embodiments may be directed to the use of ring electrodes crimped or swaged on to splines and struts, and comprising well-known materials in the art. The ring electrodes being electrically coupled to signal processing circuitry using lead wires. The ring electrodes positioned along the splines and struts form bipole pairs of electrodes with known spacing therebetween. In yet other embodiments, ring electrodes may be swaged or crimped on to a flexible circuit board comprising at least part of the splines, and/or struts of the various catheters disclosed herein.

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1-10. (canceled)

11. A basket catheter comprising:
an elongate catheter shaft comprising a proximal end and a distal end;
a flexible basket comprising a plurality of splines having a plurality of electrodes coupled to the distal end of the catheter shaft and configured to conform to tissue, the plurality of electrodes further comprising:
a plurality of mapping electrodes disposed on the plurality of splines, wherein the plurality of electrodes are configured and arranged to detect electrophysiological characteristics of tissue in contact with the basket;
at least one ablation electrode configured and arranged to selectively ablate the tissue, wherein the at least one ablation electrode is relatively larger than each of the plurality of mapping electrodes disposed on the plurality of splines; and
controller circuitry communicatively coupled to the plurality of electrodes and configured to receive signals from the plurality of mapping electrodes indicative of the electrophysiological characteristics of the tissue in contact with the flexible basket and generate an electrophysiological map of the tissue and configured to operate one or more pairs of the plurality of electrodes in one or more of a bipolar configuration and a monopolar configuration to affect an ablation therapy of the tissue based at least in part on the electrophysiological map of the tissue, wherein affecting ablation therapy comprises applying a voltage differential between 400 and 4,000 volts to produce irreversible electroporation.

12. The basket catheter of claim 11, wherein the plurality of electrodes on the plurality of splines are further configured to conduct radio-frequency tissue ablation, and to operate in both monopolar and bipolar configurations for tissue ablation.

13. The basket catheter of claim 11, wherein the plurality of mapping electrodes are weighted toward a distal end of flexible basket.

14. The basket catheter of claim 11, wherein one or more of the plurality of mapping electrodes are spot electrodes and the plurality of splines comprise flexible electronic circuit boards that are communicatively and mechanically coupled to the plurality of electrodes.

15. The basket catheter of claim 11, wherein the basket catheter further comprises a plurality of temperature sensors, each of the temperature sensors mechanically coupled to the splines and placed in thermal communication with at least one of the plurality of electrodes.

16. The basket catheter of claim 15, wherein the controller circuitry is further configured to control power delivery to each of the plurality of electrodes based at least in part upon a temperature measured in proximity to each of the plurality of electrodes by the temperature sensors.

17. The basket catheter of claim 11, wherein the plurality of electrodes comprises bipole electrode pairs that extend diagonally across adjacent splines of the basket.

18. The basket catheter of claim 11, further comprising at least one sensing electrode for determining location and/or orientation of the basket catheter.

19. The basket catheter of claim 11, wherein the controller circuitry is further configured and arranged to minimize current draw to the plurality of electrodes and deliver a desired voltage gradient.

20. The basket catheter of claim 11, wherein the plurality of electrodes comprises bipole electrode pairs that comprise electrodes on adjacent splines.

21. An electroporation ablation system for treating target tissue in a patient, the electroporation ablation system comprising:
a catheter comprising an electrode assembly, the electrode assembly comprising:
one or more ablation electrodes configured to generate electrical fields proximate to a target tissue and selectively ablate the target tissue, the one or more ablation electrodes configured to operate in one or more of a bipolar configuration and a monopolar configuration, wherein selectively ablating the target tissue comprises applying a voltage differential between 400 and 4,000 volts to produce irreversible electroporation; and
one or more mapping electrodes configured to measure electrical signals, wherein the one or more ablation electrodes are relatively larger than each of the one or more mapping electrodes.

22. The ablation system of claim 21, wherein the one or more mapping electrodes are weighted toward a distal end of the electrode assembly.

23. The ablation system of claim 21, wherein the electrode assembly is a basket assembly comprising a plurality of splines.

24. The ablation system of claim 21, wherein the one or more ablation electrodes are further configured to conduct radio-frequency tissue ablation, and to operate in both monopolar and bipolar configurations for tissue ablation.

25. The ablation system of claim 21, wherein the one or more ablation electrodes are further configured and arranged to operate the electrodes in either a monopolar or bipolar configuration during ablation therapy, depending upon desired lesion characteristics at each electrode.

26. The ablation system of claim 21, further comprising at least one sensing electrode for determining location and/or orientation of the catheter.

27. The ablation system of claim 23, wherein the plurality of splines comprise flexible electronic circuit boards that are communicatively and mechanically coupled to the one or more ablation electrodes and/or the one or more mapping electrodes.

28. The ablation system of claim 23, wherein the electrode assembly further comprises a plurality of temperature sensors, each of the temperature sensors mechanically coupled to the plurality splines and placed in thermal communication with at least one of the electrodes.

29. The ablation system of claim 28, further comprising controller circuity configured to control power delivery to each of the one or more ablation electrodes and/or the one or more mapping electrodes based at least in part upon a temperature measured in proximity to each of the respective electrodes by the temperature sensors.

30. The ablation system of claim 23, wherein the one or more ablation electrodes comprise bipole electrode pairs that extend diagonally across adjacent splines of the basket.
